(19) United States
(12) Patent Application Publication
(10) Pub. No.: US 2007/0021651 A1
(43) Pub. Date: Jan. 25, 2007

(54) Closing system for a natural or an artificial anus

(76) Inventor: Lothar Gobel, Wurzburg (DE)

Correspondence Address: Scott R Foster;Pandiscio & Pandiscio, 470 Totten Pond Road, Waltham, MA, 02451-1914, US

(21) Appl. No.: 10/565,316

(22) PCT Filed: Jul. 23, 2004
(86) PCT No.: PCT/EP04/08256

§ 371 Date: Jul. 13, 2006

(30) Foreign Application Priority Data
Jul. 23, 2003 (DE) 103 33 706.7

(51) Int. Cl. A61F 2/04

(52) U.S. Cl. 600/31; 128/DIG. 25; 623/23.67

(57) ABSTRACT

The invention relates to a closing system for a natural or an artificial anus, comprising an inflatable balloon having an approximately toroidal structure, composed of a plane tubular section folded in on itself, the ends of which (13, 14) extend coaxially within each other and are linked to a sleeve.

The invention is directed to a closing system for a natural or an artificial anus.

The medical management of colostomies continues to be an ongoing, daily problem. One very common method is to collect the stool in receptacles that are used in the form adhesive bags. This extracorporeal storage is associated with problems of odor nuisance, soiling nuisance and the risk of overflow.

In addition to extracorporeal collection systems, seals were developed with the objective of intracorporeal storage and subsequent deliberate emptying of the stool. Due to their difficult handling, these collection systems did not find very widespread acceptance. The problem was that a true seal could be achieved. The seals were not equal to the variable internal abdominal pressure.

People suffering from fecal incontinence have similar problems. Here the aim is to seal the anus with a suitable appliance to prevent uncontrolled defecation. Voluntary evacuation, on the other hand, must be encouraged or facilitated. Appliances used for this purpose always include a hose which is to be inserted in the anus and which, owing to its minimal flexibility, almost always causes pain and can even result in injury.

These disadvantages of the described prior art give rise to the problem initiating the invention, that of creating a closing system for a natural or artificial intestinal outlet that is of uncomplicated construction, can readily be implanted, is easy to handle and causes the smallest possible pressure load on the tissue, particularly the intestinal mucosa. Finally, the system should be inexpensive.

The solution to this problem is achieved by means of an inflatable balloon having an approximately toroidal structure, formed of a hose segment with a two-dimensional surface, which is inverted into itself, whereby its two ends extend roughly coaxially inside each other and are (each) connected to a sleeve. Advantageous improvements of the invention are contained in the dependent claims.

The balloon is fabricated preformed and therefore need not be inflated with a high overpressure, but only with a few mbars of overpressure relative to the ambient pressure. It thereby remains flexible in the inflated state and can adapt itself to natural conditions, for example can follow an abrupt bend in the intestine, etc. The pressure on the intestinal mucosa is always roughly constant and corresponds only to the internal pressure of the balloon. It is of great importance that the balloon has no guide shaft, so no such element, even one of reduced diameter, projects into the intestine. In the absence of a guide shaft, in the invention the inner wall of the torus is formed by the balloon itself; hence its high flexibility.

Both ends of the balloon are situated on one and the same side of the torus (due to the inversion of the hose), specifically on the side facing away from the interior of the person's body. There, the hose is fastened to one or more sleeves, which do not extend all the way through the balloon and are shorter than the inflated balloon or shorter than half the length of the original, not-yet-inverted hose, preferably less than one-fourth of this original (overall) length of the hose, particularly smaller than one-sixth of this length.

During use, these sleeves are preferably situated outside the body of a person, or they protrude only slightly through the abdominal wall or extend just to the sphincter muscle. Wearing comfort can be considerably increased in this way, since the anus or stoma is not perpetually stretched. By virtue of the preforming, the mutually concentric ends of the balloon or connection ports create a neck region that is tapered with respect to the balloon per se and extends for example through the anus to the ampulla recti, where the toroidally expanded balloon has room to deploy and thereby anchor itself.

The neck region itself remains flexible, thanks to its low internal pressure, and can be compressed cross-sectionally. Since one end of the hose is narrower than the other, a coaxial arrangement of the neck region leading to the balloon per se is preprogrammed once the inversion has been effected, and there even remains an annular gap that forms a flow connection from the toroidal interior of the balloon to a connection at the sleeveward end.

The trumpet shape imparted to the front end of the inflated balloon by the preforming facilitates, where applicable, the passage of fluids, stool, etc.

On the other hand, the central lumen, which does not communicate with the interior of the balloon and is therefore completely free of pressure, can be used for the insertion of tubes or hoses (drainage) and/or catheters or the like. It is advantageous in this regard that the central and pressure-free inner lumen is pressed flat by the pressure inside the torus, so that two plies of the hose are contiguous there, if the inflated balloon portion of the single-walled outlet hose that is not invaginated or rolled over is selected so that its length is greater than its diameter. The compressed inner hose of the double-hose segment then exerts a clamping pressure on an inserted object and thereby holds it fast in frictional engagement.

In addition, these contiguous plies of the hose form in the respective edge region two folds of finite radius, where—assuming that the central lumen is free, i.e., no object has been inserted—two narrow, capillary-shaped through-passages remain, so that for example an elevated internal pressure in the bowel can be dissipated in the natural way.

Because an inventive appliance is inserted only partially into the natural anus, an internal pressure working against the sphincter muscle is able to prompt the latter to react, thereby exercising it. Such exercise can be intensified by alternately inflating and deflating the balloon.

In other cases, the central lumen can be held open by means of a short, preferably permanently fixed, inserted ring segment; in such cases it is advisable to employ a sealing element, particularly separately inflatable balloons, disposed in the central lumen after this ring.

The balloon, made of a thin-walled, flexible and inflatable polymer, is prefabricated as to its outer dimensions in the inflated state. The balloon is inflated only in order to deploy the balloon envelope. The material used for the balloon allows the balloon to stretch to only a very small extent, since it is largely inelastic.

The polymer used is preferably polyurethane, a polyurethane/polyvinyl fluoride blend, or a comparable polyurethane-based material. This material is neutral, so it can have absolutely no harmful effects on the mucous membrane of the bowel.

In its simplest embodiment, the balloon is provided with a connecting hose port that is joined to the plug. Once the plug has been inserted into the abdominal wall, the balloon is deployed through a channel located in the plug and comes into contact by its outer wall with the intestinal wall. To facilitate the insertion of the balloon through the abdominal wall into the intestine, the plug is provided with a cavity in which the collapsed balloon can be housed.

The plug itself is preferably form-lockingly connected to a sealing cap that is known per se, which can be glued to the abdominal wall after the plug is inserted into the abdominal wall.

A collection bag to collect the stool can be connected to the channel of the plug.

The preferred embodiment of the inventive subject matter, however, provides that the plug comprise two sleeves able to be fitted one inside the other and that the balloon have two connecting hose ports whose mouths are each connected to a respective one of the sleeves. It is favorable in this case if the one mouth has a diameter adapted to the outer sleeve and the other mouth a diameter adapted to the inner sleeve. Both mouths can be glued to the sleeve walls. The mouth joined to the outer sleeve is then fastened to the outer wall of the sleeve, whereas the mouth joined to the inner sleeve is glued to the inner wall of the inner sleeve.

To form the cavity on the plug, the inner sleeve is implemented as shorter than the outer sleeve, so that the cavity present in this region suffices to house the collapsed balloon.

In further development, the inner sleeve can be provided in its interior with a stop valve. This can be a check valve that keeps fluid in the obturating bladder. In addition, a carbon filter implemented as gas-permeable can be installed in the inner sleeve. The gases produced can be diverted by this means.

The closing system configured in this manner produces a good seal that keeps fluid from escaping to the outside. Moreover, collection bags or the like are rendered superfluous. To remove the stool, the inner sleeve can be withdrawn from the outer sleeve in a very simple manner and the balloon itself can be pulled through the opening in the outer sleeve. If the balloon is suitably dimensioned, it can serve as the collecting recipient for the stool.

For cases in which the size of the balloon is not adequate for this purpose, a special, larger collection receptacle for the stool can be used, which can be connected to the sealing cap by a first adapter and to the inner sleeve by a second adapter. Via the second adapter, the inner sleeve, which is inserted force-lockingly into the outer sleeve, can be withdrawn from the latter. It takes the balloon along with it in the process, and also withdraws the outer sleeve from the sealing cap once the balloon has been pulled all the way through. The stool can then be emptied completely into the collection receptacle.

Further features, characteristics, advantages and effects based on the present invention will be apparent from the following description of several preferred exemplary embodiments of the invention and from the drawing. Therein:

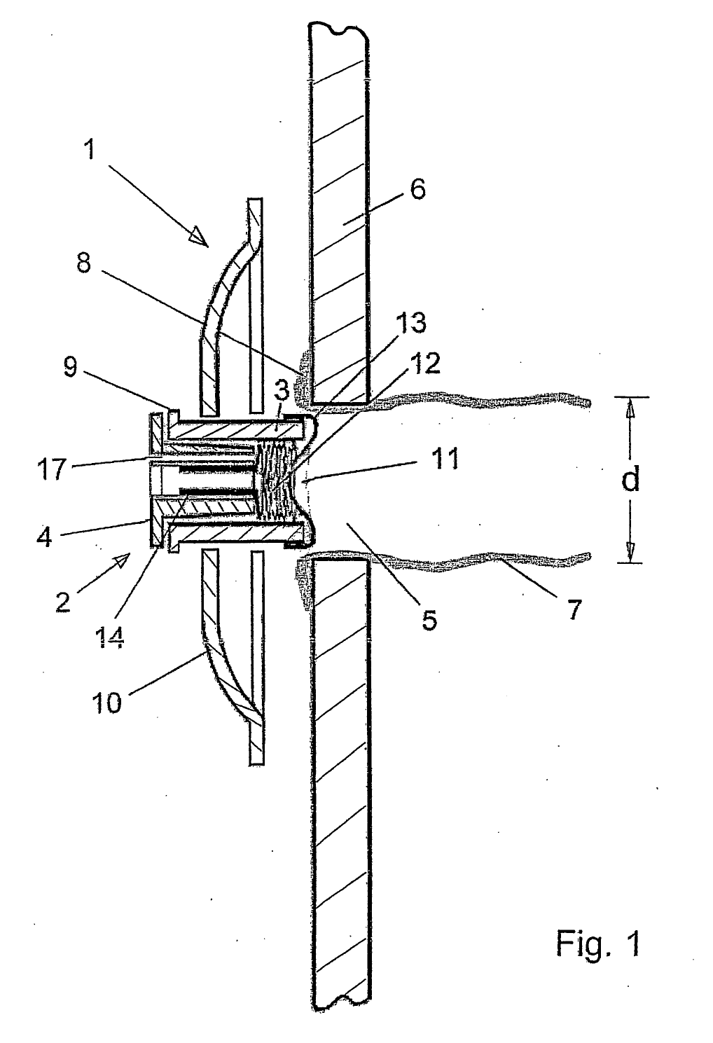

FIG. 1 is a section through an abdominal wall with the closing system in longitudinal section during the process of implantation in the opening in the abdominal wall;

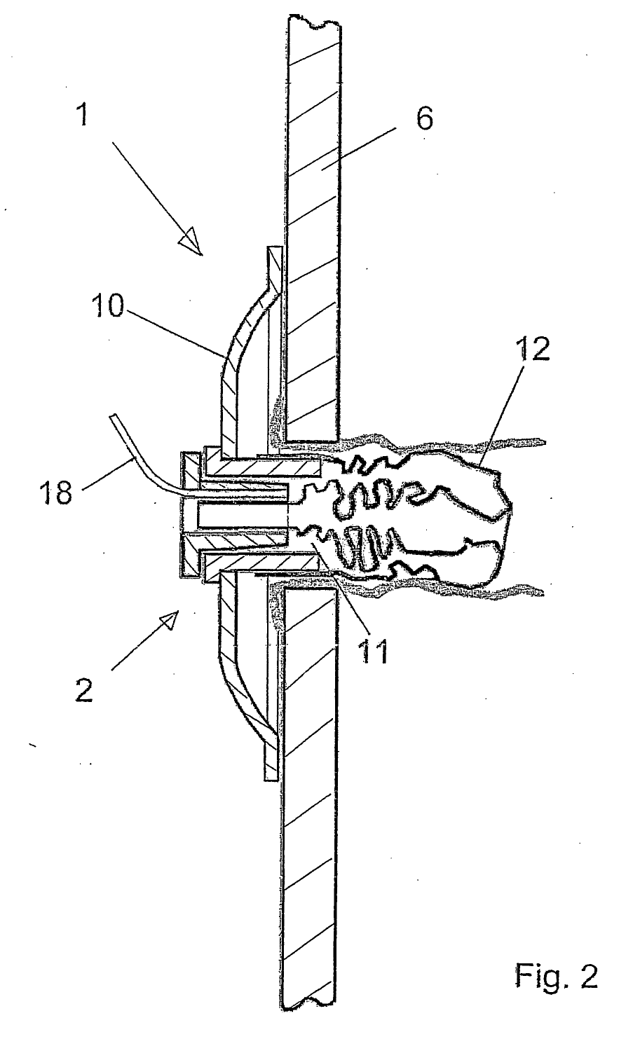

FIG. 2 shows the implanted closing system in section at the beginning of the process of deploying the balloon;

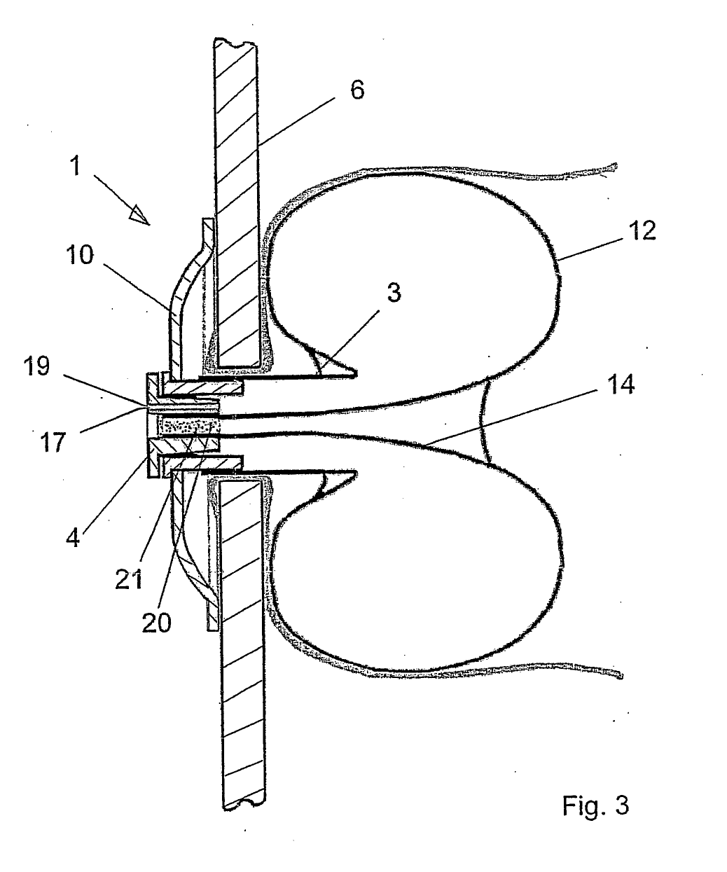

FIG. 3 shows the closing system with the balloon inflated;

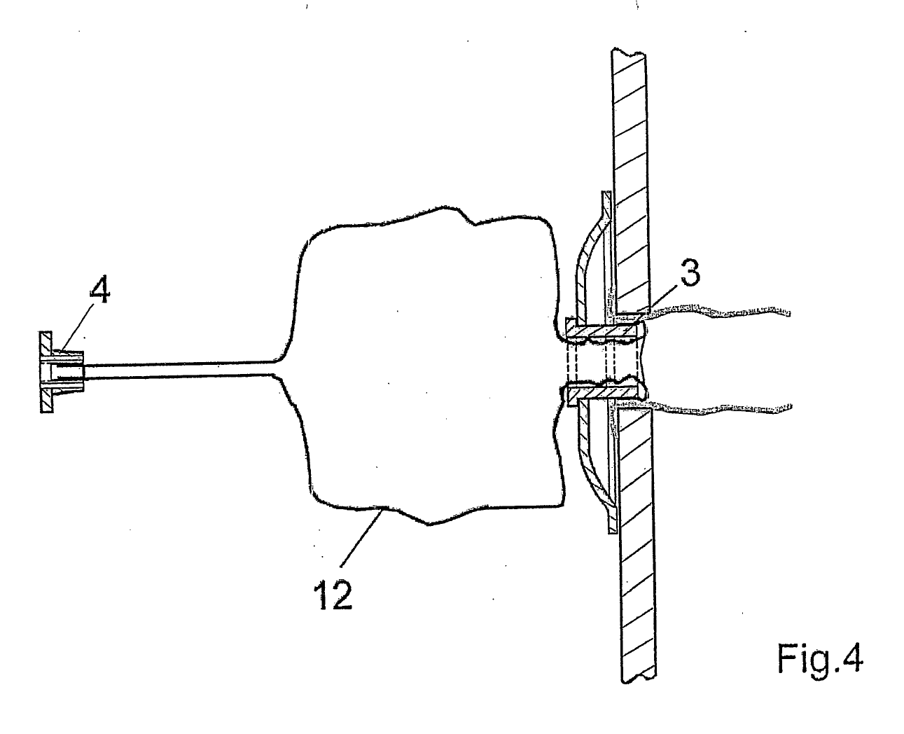

FIG. 4 shows the closing system with the inner sleeve and the balloon withdrawn;

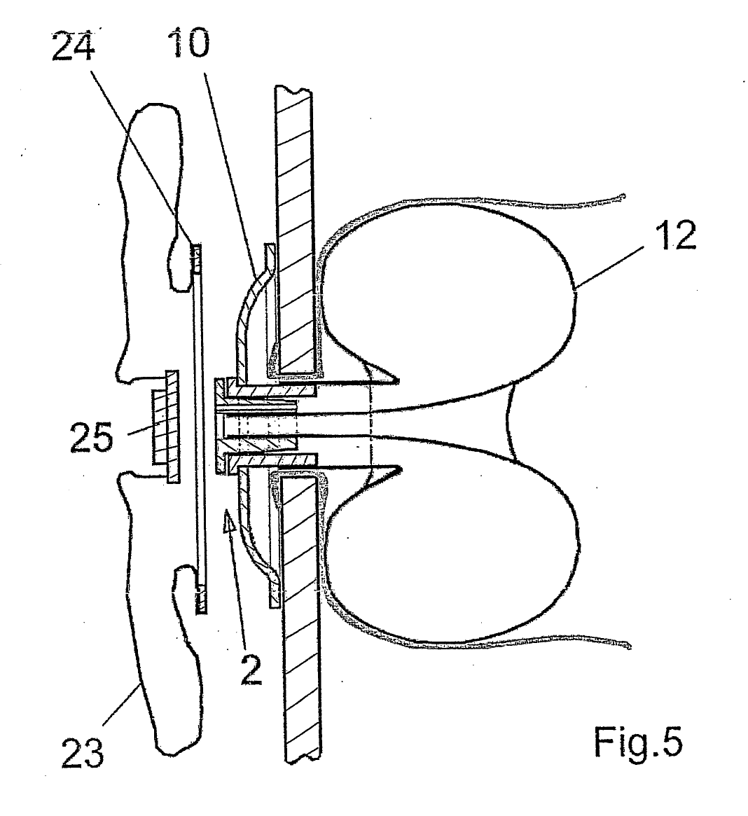

FIG. 5 shows the closing system with a collection receptacle ready to be fitted thereto;

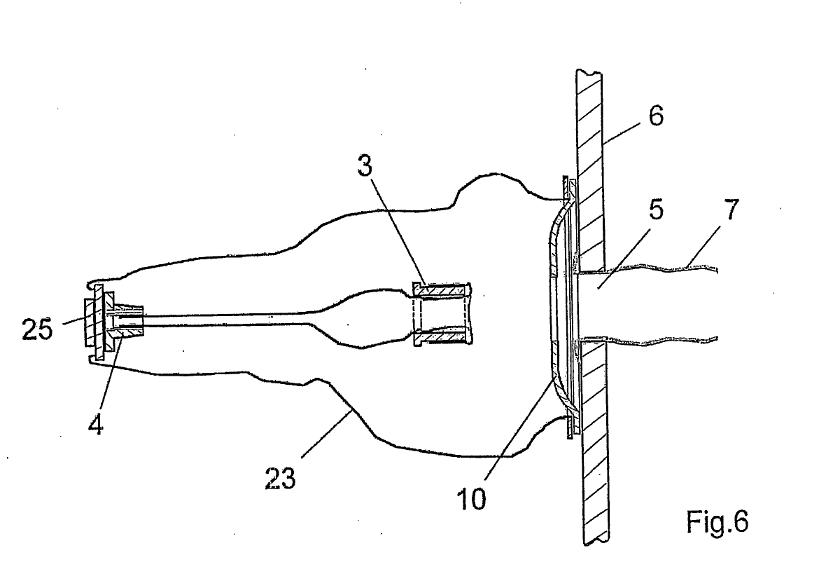

FIG. 6 shows the closing system with the inner sleeve withdrawn, including the balloon, and with the collection receptacle intended to receive the stool;

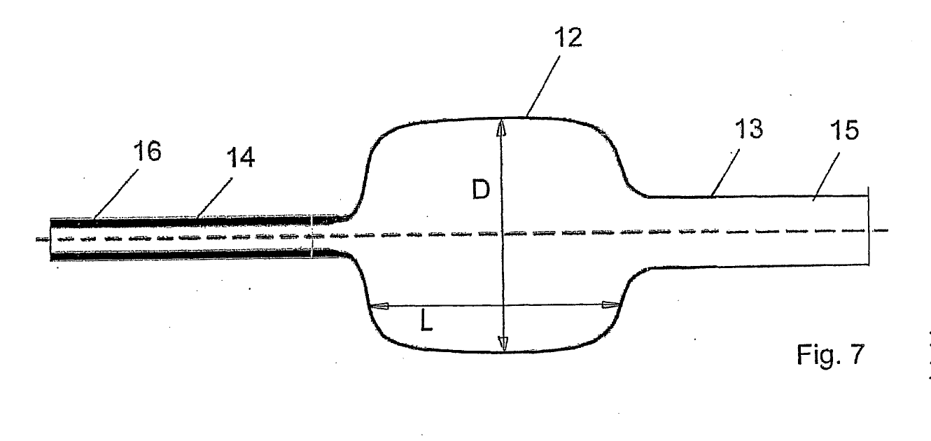

FIG. 7 is a section through the preformed balloon with hose connectors;

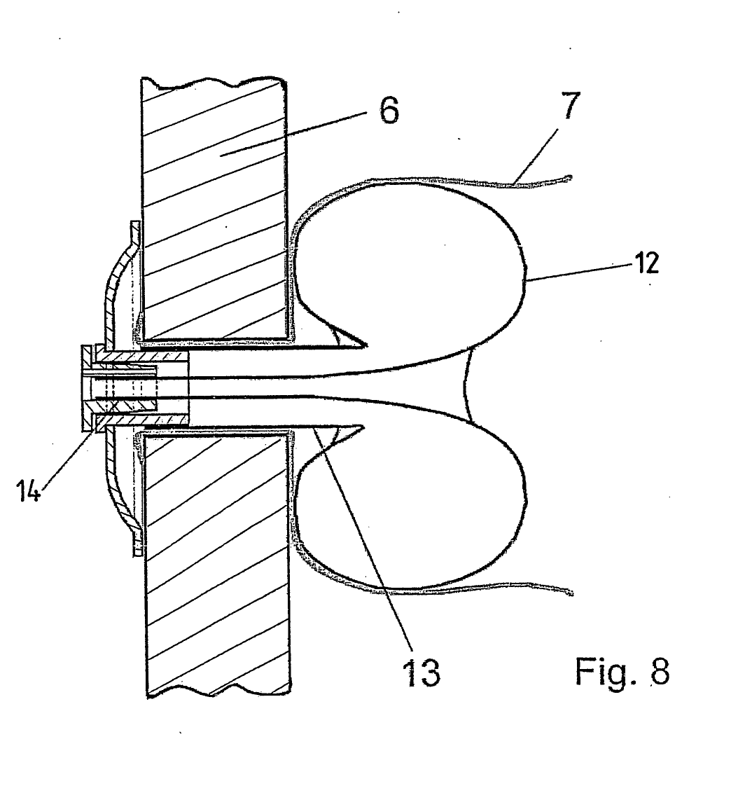

FIG. 8 shows a balloon with an elongated hose connector, implanted in a thicker abdominal wall;

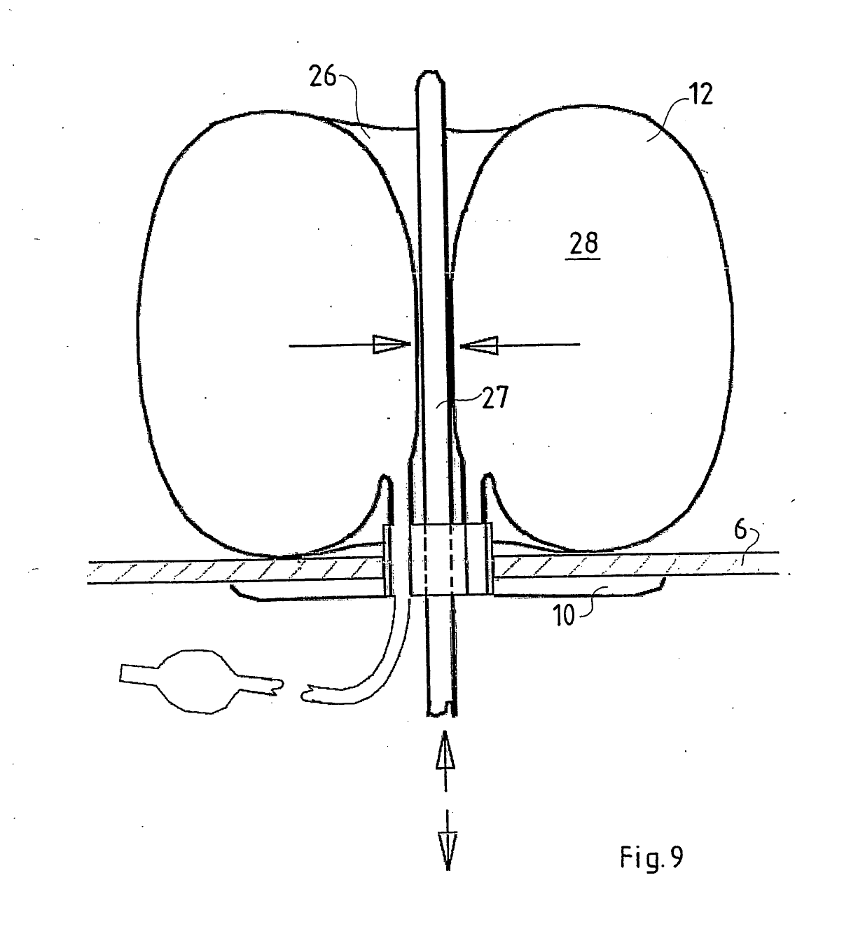

FIG. 9 shows an embodiment of the invention corresponding to the balloon from FIG. 8, with the insertion of a catheter;

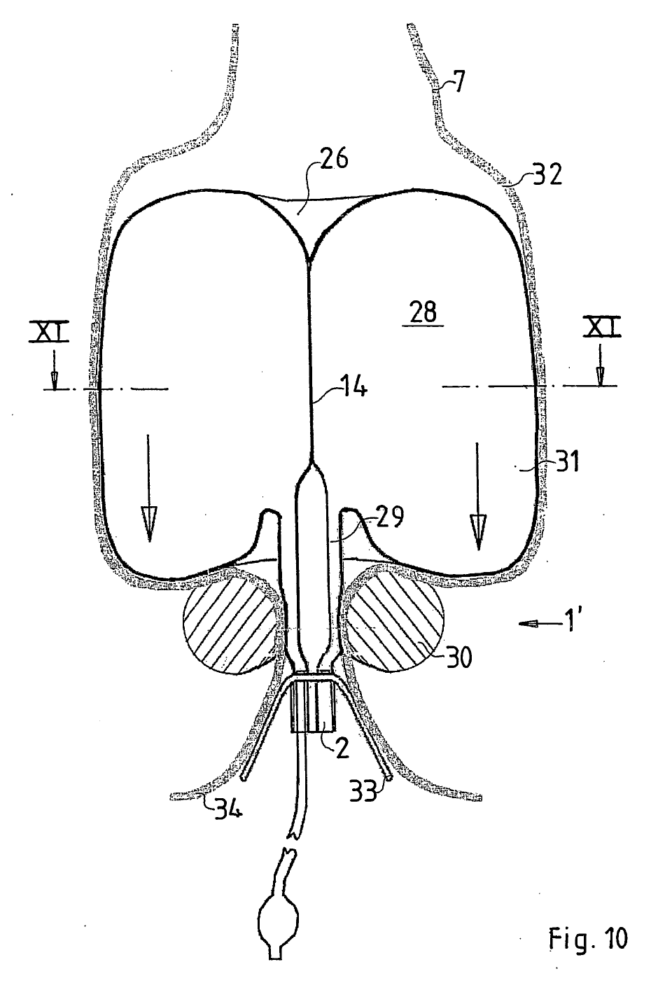

FIG. 10 shows another embodiment of the invention, optimized for use in the natural intestinal outlet;

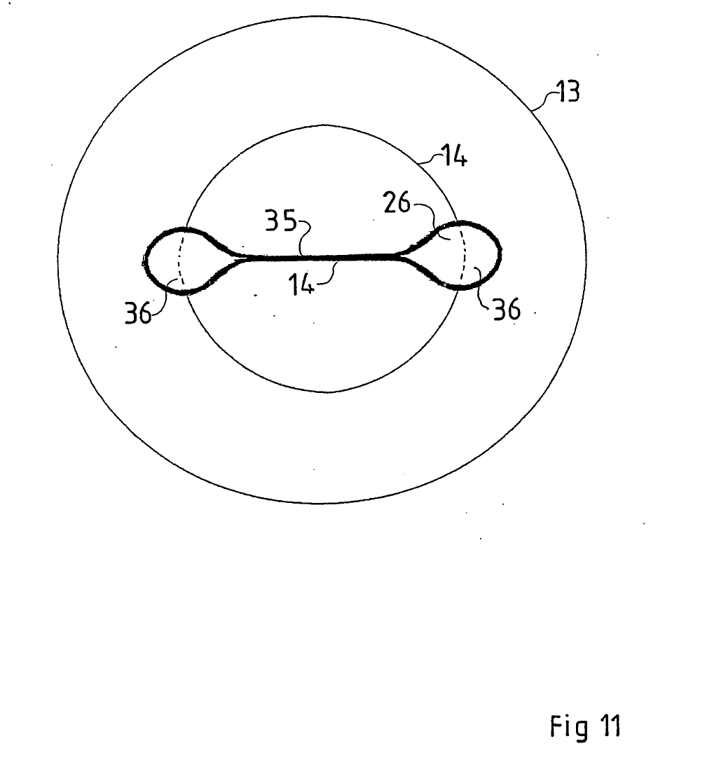

FIG. 11 is a section through FIG. 10 along line XI-XI;

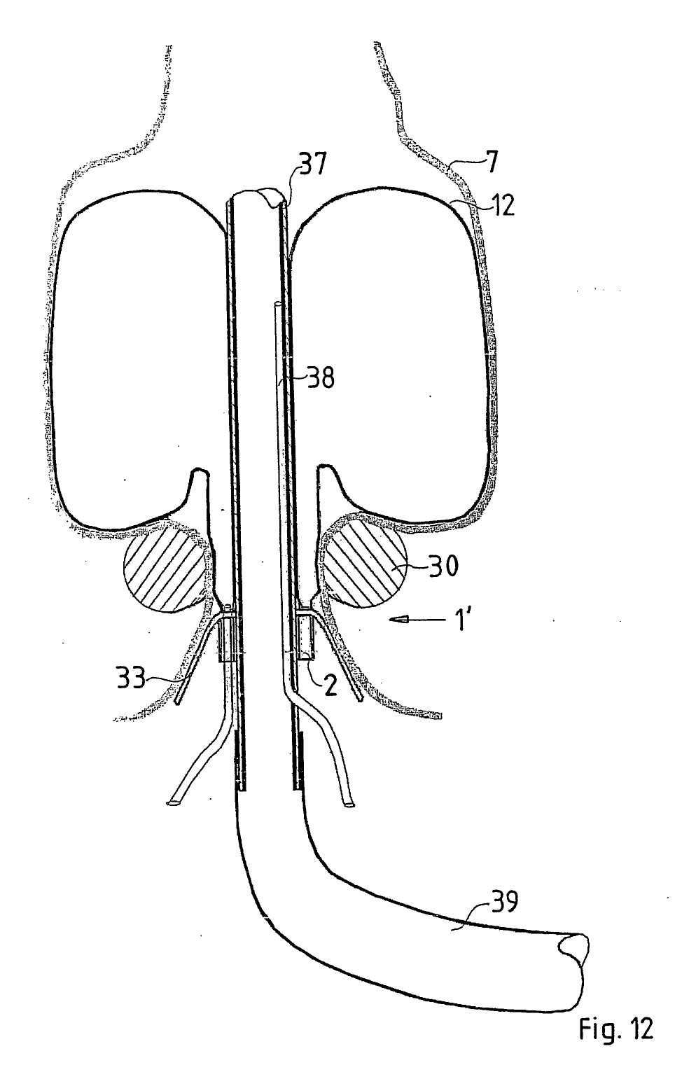

FIG. 12 shows a type of construction related to the embodiment depicted in FIGS. 10 and 11 and suitable for receiving a drainage tube;

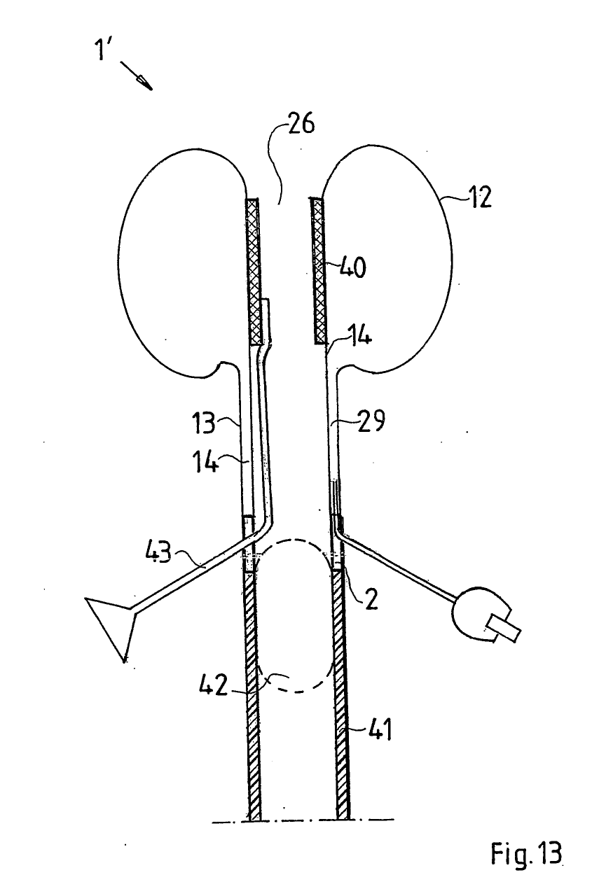

FIG. 13 depicts a further modified embodiment of the invention; and

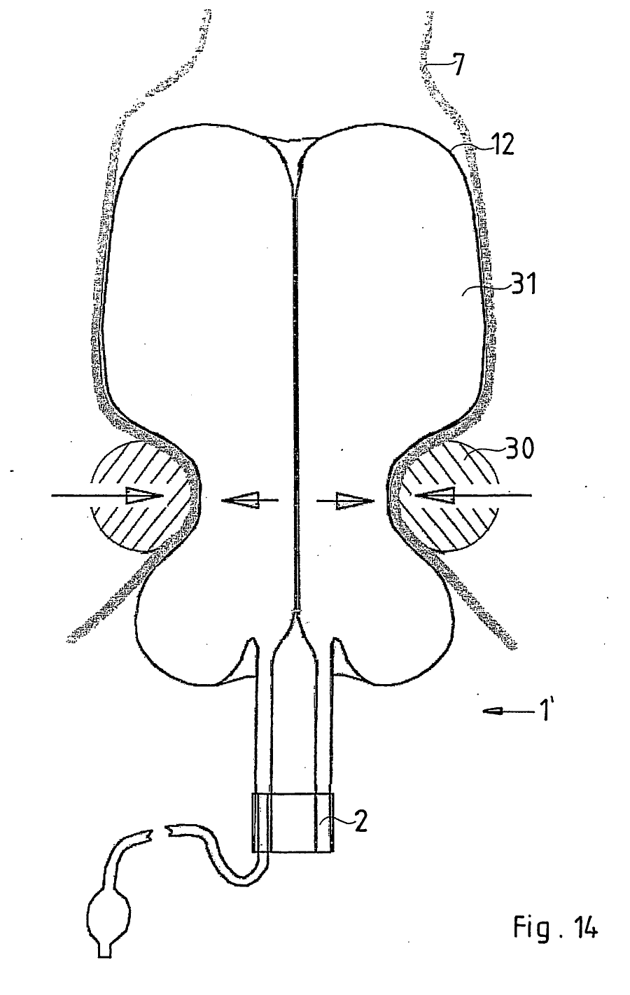

FIG. 14 depicts the use of the invention for exercising the sphincter muscle.

Represented schematically in FIG. 1 is the closing system 1 for use with a colostomy, specifically based on an embodiment in which the plug 2 is composed of two sleeves 3 and 4 that can be fitted one inside the other. Inner sleeve 4 is in adjacent contact inserted into outer sleeve 3. To this end, it is configured as slightly conical.

In the figure, the closing system 1 is shown being inserted into the opening 5 in the abdominal wall. The abdominal wall 6 is of normal configuration. The bowel 7 is sutured by its end 8 to the abdominal wall 6 in a manner that is known per se.

With its externally disposed flange 9, outer sleeve 3 grasps the sealing cap 10, which when the plug 2 is inserted completely comes into contact with the abdominal wall and can be glued thereto. It should be noted that the sealing cap 10 provides extra security for the patient in regard to the escape of body fluids. In addition, the cap protects the short segment of bowel exteriorized to the surface of the body. This segment is otherwise left unprotected against mechanical irritations. In particular, however, the sealing cap helps to prevent the drying and necrotization which at this location threatens the exteriorized bowel segment, which is devoid, here, of keratinized epithelium, i.e., natural liquid barriers. It is also sufficient per se if plug 2 or outer sleeve 3 is provided for this purpose with an enlarged annular flange 9 that covers the edge of the opening 5 and the exteriorized segment of bowel. As an abutment for the balloon inflated inside the body, the sealing cap—or the flange in the usual case—is not used if the innervation of the terminal segment of bowel has been preserved, since the propulsive movements of the bowel constantly strive to push the obturating balloon toward the outside of the body, against the inner abdominal wall.

Inner sleeve 4 is configured as shortened compared to outer sleeve 3, thereby producing a cavity 11 into which the collapsed balloon 12 can be folded. As shown in FIG. 7, the balloon 12 has two connection ports 13 and 14 by which it is connected to outer and inner sleeves 3, 4, respectively.

Balloon 12 with connection ports 13 and 14 is made of a thin-walled, inflatable polymer and has when inflated a diameter D that is appreciably greater than the diameter d of the bowel segment concerned. Diameter D is produced in various sizes and can in this way be adapted to the size of bowel diameter d. This also applies to the execution of the plug 2 and the sleeves 3, 4. In the exemplary embodiment according to FIG. 1, the larger connection port 13 is pulled by its mouth 15 onto the outer wall of sleeve 3. The mouth 16 of connection port 14 is fastened to the inner wall of inner sleeve 4. The fastening can be done with glue, but clamping rings or the like are also feasible. For the operation of inflating the balloon 12, which by virtue of its being fastened to plug 2 is configured as double-walled, channel 17 is provided in inner sleeve 4.

In FIG. 2, plug 2 is fully inserted in the opening, so that sealing cap 10 rests against the abdominal wall. Through the hose nipple 18, air is pressed into the balloon 12 so that the balloon deploys. The beginning of the deployment is particularized in FIG. 2. Therein, the balloon is already pushed partway out of the cavity 11.

FIG. 3 shows the fully inflated balloon 12, which has assumed the shape of an annular ring and rests sealingly against the wall of the bowel 7. The preforming of the balloon 12 during manufacture reflects its shape when inflated. The annular ring can be of different lengths, so that it is also configured as cylindrical and occupies a longer segment in the bowel 7.

The deployed balloon 12 is configured with respect to its diameter D such that it is larger than the maximally distended bowel, so that excess balloon wall material of the outer hose, when inflated, lies in folds, which due to the very small wall thickness form fold “eyelets” roughly the size of capillaries. Fluids are therefore retained in the fold eyelets and the pressure measured externally via the channel 17 corresponds to the pressure exerted on the intestinal mucosa, since it is not added to by the wall tension of the material. The pressure on the intestinal wall 7 is therefore sufficient for sealing, although the risk of infarction of the bowel cannot be averted completely in this way. A factor that is favorable for sealing is that the annular ring also bows outward toward the abdominal wall 6 and there presses sealingly against the bowel 7 on the inside of the abdominal wall 6.

Installed in the air channel 17 of inner sleeve 4 is a check valve 19 that keeps the air in the balloon 12. This valve can be opened if necessary and the air vented. Connection port 14 effectively forms an inner wall of the balloon 12, which constitutes an escape channel 20 for the gases produced in the bowel 7. Installed in this channel 20 or in inner sleeve 4 is a carbon filter 21 that prevents liquid stool from escaping through said channel 20.

To evacuate the bowel, it is possible in many cases to let the air out of the balloon 12 or open valve 19 or withdraw inner sleeve 4 from outer sleeve 3. Withdrawing the inner from the outer sleeve causes the obturating balloon to lose pressure and thus deflate. The entire balloon 12 can then be pulled through the inside of outer sleeve 3. The then externally disposed balloon 12 can receive the stool. After cap 10 with outer sleeve 3 has been detached from the abdominal wall 6, the stool can thus be removed easily and safely.

FIG. 4 shows the position of the balloon 12 in which it has already been pulled through outer sleeve 3 and is ready to receive the stool.

Since the balloon 12 will not be adequate to receive relatively large amounts of stool in every case, it is possible to configure the plug 2 and/or the sealing cap 10 such that an appropriately configured collection bag 23 can be fastened thereto. The collection bag 23 has an annular flange 24 that can be connected to sealing cap 10 and a lid 25 that can be placed on inner sleeve 4. By the exertion of traction on lid 25, inner sleeve 4 is withdrawn from outer sleeve 3 and, as represented previously in FIGS. 3 and 4, balloon 12 is withdrawn through the inner opening of outer sleeve 3. This procedure is illustrated in FIG. 6, wherein outer sleeve 3 is also pulled out of opening 5 or its mounting in the cap 10, so that the stool can be emptied into the collection bag 23.

FIG. 7 shows the preformed balloon 12 with the connection ports 13 and 14. Connection ports 13 and 14 have a relatively great length. Before they are used, i.e. connected to plug 2 or sleeves 3 and 4 of plug 2, connection ports 13 and 14 are custom-cut to an appropriate length, depending on the thickness of the abdominal wall 6.

FIG. 8 shows the implantation of a balloon 12 in association with a thicker abdominal wall 6, all other parts being the same as those illustrated in FIG. 3. The connection ports 13 and 14 have merely been left longer in keeping with the abdominal wall 6, so that the non-deployable portion of balloon 12 constituted by connection ports 13 and 14 still reaches into the bowel 7.

Attention should be paid to the fact that the length of the sleeves 3, 4 and of the plug 2 formed therefrom is roughly equal to or only slightly greater than the thickness of the abdominal wall 6 and therefore—due also to the depth of sealing cap 10—barely extends into the bowel 7. The subsequent course of the bowel 7 is therefore completely arbitrary; it can even kink immediately beneath the abdominal wall 6.

From the arrangement depicted in FIG. 9, it can be seen that the central lumen 26 inside the roughly toroidally inflated balloon 12 is also particularly well suited for the insertion of a catheter 27. In this case, the slight overpressure inside the balloon volume 28 presses approximately radially inward against the central lumen 26 and clamps the inserted catheter 27 firmly, thereby making it tight. The catheter 27 can be permanently fixed in the region of the sleeve segment disposed outside the body and can be provided with a suitably atraumatically shaped tip and a drainage opening for the venting of intestinal gas. The catheter fixed in the seal is fashioned with respect to its shaft mechanics such that when the seal is inserted in the body, the catheter carries the sealing balloon collapsed on the catheter shaft without bending and thereby facilitates transanal passage of the seal. It preferably measures only a few millimeters (app. 2-4 mm) in diameter. Its tip protrudes only slightly beyond the distal end of the filled balloon body.

FIGS. 10 to 14 below reflect embodiments that are suitable for use with a natural intestinal outlet.

The closing system 1′ used in this case differs only in detail from those described hereinabove. For example, the connection ports 13, 14 can be configured as somewhat longer, thereby resulting in a pronounced neck region 29 that extends through the sphincter muscle 30 and makes it possible for the actual, radially expanded balloon portion 31 to fill the ampulla 32. Since when the balloon 12 is inflated its radially expanded portion 31 is pressed against the floor of the ampulla 32, this closing system 1′ is able to anchor itself in optimum fashion. The abutment is formed in this case by a longitudinally folded sealing cap 33 that is fastened to plug 2 and whose shape is adapted to the anatomy of the anal fold 34. The sealing cap 33 can be provided with a soft fleece on the outer sides of its two wings.

As a side effect of inflation, the balloon volume 28 also presses against the central lumen 26 and in so doing collapses inner hose segment 14, as indicated in FIG. 11 by the thick line. The central lumen 26 is thereby largely sealed. Nevertheless, due to the limited deformability of the thicker hose material on the inner hose segment 14, capillary-shaped passages 36 do remain on both sides of the double-ply region 35 and permit the escape of gases, but not liquids.

FIG. 12 shows that this embodiment 1′ is also suitable for the insertion of a drainage tube 37 by means of which flushing of the bowel 7 can be effected. For this purpose, a liquid, for example water, is conveyed into the bowel 7 by means of a hose 38 guided through this tube 37. To carry off the outflowing liquid, a hose 39 is connected to, particularly slipped over, the external end of the drainage tube 37.

With the closing system 1′ of FIG. 13, the natural intestinal outlet can be sealed, but normal evacuation of the bowel can also be brought about as necessary. For this purpose, the central lumen 26 is held constantly open by a ring or a short tube segment 40, which is fixed to inner hose segment 14 only in punctiform or linear fashion. This ring or short tube segment 40 is shorter than the axial extent of the radially expanded balloon portion 31. It is connected to the also ring-shaped plug 2 only via the neck portion of balloon 12 formed by connection ports 13, 14. Attached at the far end of this ring or plug 2 is a hose segment 41. The rings 2, 40 and hose segment 41, which is more rigid than balloon 12, hold central lumen 26 continuously open, so that spontaneous defecation is possible. In order, conversely, to control and even suppress or postpone such defecation, there is provided in central lumen 26, in neck segment 14, [in] one of the rings 2, 40 and/or in the hose segment 41 an influencable sealing element in the form of a second, separately inflatable balloon 42, which for example can be affixed by means of glue or the like to the inner face of the segment 2, 14, 26, 40, 41 concerned and can be filled or emptied via a separate line. To be able to also stimulate defecation, an additional line 43 is provided, which for example passes through the plug or ring 2 into the central lumen 26 and can be anchored by its end for example to the front ring 40. Through this line 43, for example water or another liquid can be introduced in order to flush out the bowel.

If in this embodiment 1′ the length L of the balloon region 31 that is preformed to the shape of the abdomen is shorter than its outer diameter D, then—given a moderate overpressure inside the balloon 12—ring 40 is not necessary to keep the central lumen 26 open, because in this case a toroidal shape is created that is nearly ideal and is therefore always open at the center 26.

Another application for the inventive closing system 1′ is illustrated in FIG. 14. Here, the balloon 12 is not inserted completely into the bowel 7, but only partially, so that it is located just at the level of the sphincter muscle 30. Then, by variably raising and lowering the pressure inside the balloon volume 28, the sphincter muscle 30 can be stretched and an opposite closing reflex can be elicited. By repeating this process, the sphincter muscle 30 can be exercised regularly to actively reduce fecal incontinence. To better guard against dislocation of the balloon 12 during normal physical movement of the patient (walking, sitting), the balloon can in the transanal segment be suitably preformed with a taper, or waisted (about 2-5 cm in diameter transanally).

To make the sealing apparatus usable for the self-care or self-initiation of hemorrhoidal bleeding by the patient, the body can be supplemented by an initiating and drainage element similar to that shown in FIG. 9, preferably permanently fixed in the sleeve terminating segment.

What is claimed is:

1. A closing system for a natural or artificial anus, comprising an inflatable balloon having a generally toroidal structure, formed of a hose segment with a two-dimensional surface, which hose is inverted into itself, whereby its two ends (13, 14) extend generally coaxially with each other and are connected to a respective sleeve.

2. The closing system in accordance with claim 1, wherein said balloon (12) is preformed with two connection ports (13, 14) or ends.

3. The closing system as recited in claim 2, characterized in that said connection ports (13, 14) or ends of said balloon (12) are preformed such that each or both of end regions (13, 14) of the inverted hose segment have a generally constant cross-sectional length.

4. The closing system in accordance with claim 3, wherein said connection ports (13, 14) or ends of said balloon (12) are preformed such that cross sections through the two ends of the inverted hose have different lengths that correspond to different circumferential lengths of said end regions.

5. The closing system in accordance with claim 1, wherein the inverted hose is preformed such that its front end, which is distal relative to the mutually coaxial ends, assumes in an inflated state a gently curved contour with no edge regions.

6. The closing system in accordance with claim 2, wherein said balloon is preformed such that it has in an inflated state a diameter that exceeds a diameter of a bowel segment.

7. The closing system in accordance with claim 6, wherein the length of the sleeve(s) is in each case smaller than one third the length of the balloon measured coaxially to the axis of symmetry of the inflated balloon.

8. The closing system in accordance with claim 6, wherein the collapsed balloon (12) is housed in a cavity (11), which is provided in a plug (2) and is directed toward an interior of the bowel.

9. The closing system as recited in claim 8, characterized in that two connection ports (13, 14) of said balloon (12) are each connected by their mouths to said plug (2).

10. The closing system as recited in claim 8 wherein said plug (2) comprises two sleeves (3, 4) that can be fitted one inside the other and in that a mouth (15, 16) of each connection port (13, 14) is connected to a respective one of said sleeves (3, 4).

11. The closing system as recited in claim 10, wherein the mouth (15) that is connected to an outer (3) of the sleeves has a diameter adapted to the outer sleeve (3), and the mouth (16) that is connected to the inner (4) of the two sleeves has a diameter adapted to the inner sleeve (4).

12. The closing system as recited in claim 11, wherein said balloon (12) can be pulled through the outer sleeve (3).

13. The closing system as recited in claim 11 wherein the inner sleeve (4) comprises an air channel (17).

14. The closing system as recited in claim 13, characterized in that said air channel (17) comprises a stop valve (19).

15. The closing system as recited in claim 11, wherein a carbon filter (21) can be disposed inside the inner sleeve (4).

16. The closing system in accordance with claim 10, wherein said plug (2) and/or one or more sleeves is/are connectable to a sealing cap (10).

17. The closing system as recited in claim 16, characterized in that said sealing cap (10) is connected in adjacent contact to said plug (2).

18. The closing system as recited in claim 16, wherein said sealing cap has a folded structure.

19. The closing system in accordance with claim 16, wherein said sealing cap (10) and/or said plug (2) is connectable to a collection bag (23).

20. The closing system in accordance with claim 19, wherein the collection bag (23) is connectable to said sealing cap (10) and to the inner sleeve (4).

21. The closing system in accordance with claim 1, wherein said balloon is made of a thin-walled polymer.

22. The closing system in accordance with claim 21, wherein said polymer is a selected one of polyurethane, a polyurethane/polyvinyl chloride blend and a comparable polyurethane-based material.

23. The closing system in accordance with claim 1, wherein a ring-shaped element is fixed in a central lumen of the hose segment inverted into itself, the fixing being effected only along a narrow, circumferentially surrounding line so as not to deteriorate freedom of movement of the balloon.

24. The closing system in accordance with claim 23, wherein an externally controllable sealing element in the form of a separately inflatable balloon is disposed in the central lumen of the hose segment inverted into itself.

25. The closing system in accordance with claim 24, wherein a tube can be inserted through the central lumen of the hose segment inverted into itself.
